(19) United States
(12) Patent Application Publication
(10) Pub. No.: US 2002/0146412 A1
(43) Pub. Date: Oct. 10, 2002

(54) Method of treating patients with bacterial infections

(76) Inventors: James Brady, Riverdale, NY (US); Vadim Davankov, Moscow (RU)

Correspondence Address: Ilya Zborovsky, 6 Schoolhouse Way, Dix Hills, NY, 11746, US

(21) Appl. No.: 09/829,252

(22) Filed: Apr. 10, 2001

(51) Int. Cl. A61K 39/395

(52) U.S. Cl. 424/140.1

(57) ABSTRACT

A method of treating serious infections and sepsis includes withdrawing blood from a patient, passing the withdrawn blood through a hemocompatible particulate polymeric material which includes a first group of particles which are charged so as to provide adherence of endotoxin to the hydrophobic inner surface of particles of the first group, and also a second group of particles which are not charged and have a pore size selected so that cytokines and superantigens adhere to the hydrophobic inner surface of particles of the second group, and thereafter returning the blood to the patient.

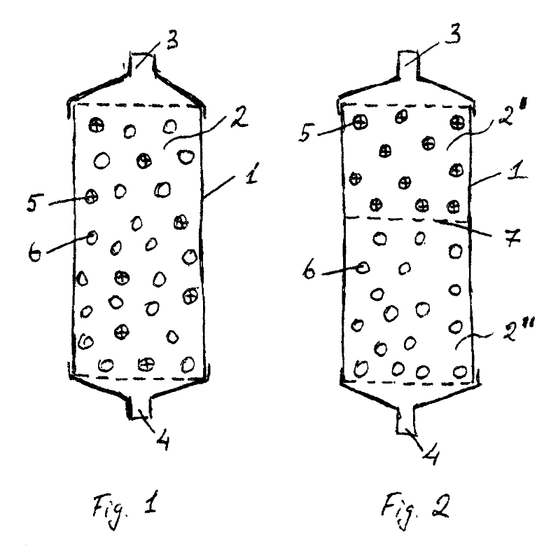

### BACKGROUND OF THE INVENTION

The present invention relates to a method for treating patients with bacterial infections that may lead to a variety of “sepsis syndromes”, shock, organ failure and death.

Infections from bacteria are responsible for many deaths each year. Bacteria are generally divided into two classes, called Gram-positive and Gram-negative, because of differences in their outer cell membranes. Both classes of bacteria are capable of causing serious illness and death due to the production of toxins that poison the body. Patients with Gram-negative infections can develop a condition called septic shock that is characterized by high fever, low blood pressure and multiple organ failure. Septic shock is fatal in over 50% of cases, even with the use of antibiotics. Patients with Gram-positive infections can develop gastrointestinal food poisoning, toxic shock syndrome, Gram-positive sepsis, and septic shock. Serious Gram-positive infections can produce shock and multi-organ failure soon after the onset of symptoms, and are associated with a mortality of up to 80%.

Severe infections leading to organ dysfunction and sepsis occur in approximately 750,000 U.S. patients each year, resulting in at least 225,000 deaths. Annual costs in the U.S. associated with septicemia and septic shock range up to $10 billion per year. Worldwide, sepsis affects millions of patients, costing many billions of dollars.

Gram-negative bacteria produce a very potent toxin called endotoxin or lipopolysacchride (LPS). LPS is a component of the cell membrane and each bacterium has over 350,000 molecules of LPS on its surface. The release of LPS into the blood stream in a patient with a Gram-negative infection can cause fever, low blood pressure and organ failure.

In serious Gram-negative and Gram-positive infections, bacteria and the toxins they produce enter the bloodstream, causing massive activation of the body's immune system. LPS, from Gram-negative bacteria, and a group of toxins called superantigens, from Gram-positive bacteria, are both potent activators of the immune system. In response to LPS and superantigens, white blood cells secrete a class of hormone-like proteins, called cytokines, which further activate the immune system and other organs to fight the infection. In septic shock and toxic shock syndrome, huge amounts of cytokines are made and overcome the body's capacity to eliminate them. High levels of cytokines can have direct toxic effects on the organs and contribute to multiple organ failure and death. Animal and human studies demonstrate that the simultaneous presence of high levels of LPS and cytokines are associated with a poor clinical outcome (reviewed by Malchesky PS, Zborowski M, Hou KC, Extracorporeal techniques of endotoxin removal: a review of the art and science, Adv Ren Replace Ther 1995 Jan;2(1):60-9)

In blood and aqueous solutions, individual molecules of LPS coalesce into vesicles ranging in size from 300,000 to 1,000,000 daltons. Phosphoryl groups contained within LPS give it an overall negative charge at physiological pH. In contrast, bacterial superantigens, which range in size from 22,000 to 29,000 daltons, are low molecular weight proteins. Cytokines are also low molecular weight proteins, ranging in size from 8,000 to 28,000 daltons. Unlike LPS, superantigens and cytokines exist in blood either as monomers or small oligomers or bound to other carrier proteins. Superantigens and cytokines are both neutral proteins with no dominant charge at physiological pH.

In the early stages of an infection, it is often very difficult to tell whether the patient is suffering from a Gram-negative or Gram-positive infection. This decision is critical because it determines what type of treatment, including the choice of antibiotic, which should be used. Irrespective of the type of infection, removing LPS, cytokines and superantigens that all have toxic effects on the body, could be a major therapeutic approach for treating patients with serious infections.

Patients with serious infections are usually treated in an intensive care unit with antibiotics and a variety of blood purification devices. The most prevalent technique uses membranes to hemodialyze and/or hemofilter the blood, either intermittently or continuously during the course of the illness. A recent clinical study of hemofiltration in patients with sepsis showed that adsorption, not filtration, appeared to be the main clearance mechanism for cytokines. Aggregates of LPS are also not filtered due to their large size. The surface area of a hemofilter is small, 0.5 m², and is rapidly saturated within the first hour of therapy. (De Vriese A S, Colardyn F A, Philippe J J, Vanholder R C, De Sutter J H, Lameire N H, Cytokine removal during continuous hemofiltration in septic patients, J Am Soc Nephrol 1999 Apr;10(4):846-53)

Hirai et al. (EP 0 800 862 A1, 1995) described the ability of a sulfonated polystyrene-type cation exchanger Diaion HPK-55H to adsorb some of these toxins from physiological saline, including endotoxin, tumor necrosis factor-60 and several additional cytokines. Macroporous resins, such as XAD-7, have also been tested for their ability to remove endotoxin and cytokines from solutions. While XAD-7 was effective in adsorbing cytokines, it was incapable of adsorbing endotoxin from human plasma (Nagaki M, Hughes R D, Lau J Y, Williams R, Removal of endotoxin and cytokines by adsorbents and the effect of plasma protein binding, Int J Artif Organs 1991 Jan;14(1):43-50) A more selective approach for endotoxin removal from blood is achieved by covalently bonding Polymyxin-B, an antibiotic that adsorbs endotoxin, to the surface of fibers contained in a device housing. Polymyxin B adsorbs LPS through a lipophilic interaction with Lipid A, one of the principal components of LPS, and through ionic attraction of LPS's negatively-charged phosphoryl groups. (see review by B. L.Jaber et al., Extracorporeal Adsorbento-Based Strategies in Sepsis, American Journal of Kidney Diseases, Vol. 30, No 5, Suppl. 4, 1997, pp S44-S56). None of the previous art, however, attempted effective, simultaneous removal of the complex pool of toxins associated with serious infections and sepsis.

### SUMMARY OF THE INVENTION

Accordingly, it is an object of the present invention to provide a method of treatment of patients with serious infections which efficiently prevent and/or treats septic shock.

In keeping with these objects and with others which will become apparent hereinafter, one feature of the present invention resides, briefly stated, in a method of treating patients with bacterial infections, in accordance with which blood is passed through a bed of porous polymeric particles which have a hydrophilic hemocompatible outer surface and positively charged groups on the hydrophobic surface of inner macropores so that endotoxins adhere to an inner surface of the charged polymeric particles, and also passes through uncharged particles which are hydrophobic in their interior and have pore sizes, such that cytokines and superantigens penetrate to the pores and adhere to the uncharged particles.

When the method is performed in accordance with the present invention, endotoxins, cytokines and superantigens are removed from blood when blood passes through the above-mentioned material with charged and uncharged particles, and therefore blood is purified from endotoxins, cytokines and superantigens, so that septic shock is reliably prevented.

### DESCRIPTION OF THE PREFERRED EMBODIMENTS

In accordance with the present invention, in order to prevent and/or treat serious infections and sepsis, blood is withdrawn from the patient, is purified by passing it through a hemocompatible blood purifying particulate polymeric material and then is returned back to the patient.

The particulate polymeric material includes first a group of polymer particles composed of a hydrophilic coating or shell to provide biocompatibility, and also a hydrophobic porous core to which endotoxin binds. Endotoxin molecules form aggregates in aqueous media, such as blood, ranging from 300 to 1,000 kDa. In order to provide a reliable interaction between endotoxin and the polymer interior, the polymer particles have pores of a corresponding large size. For example, the size of the pores can be within the range of 20 to 150 nm, preferably between 30 and 100 nm. The polymeric particles of the first group are thus predominantly macroporous.

In addition, the polymer particles can also have positively charged functional groups placed on the surface of the hydrophobic pores to further attract endotoxin through an ionic interaction. The amount of these positively charged groups should remain low, preferably below 1 meq/ml, in order not to compromise the overall hydrophobic nature of the core of the polymeric particle, so that hydrophobic interactions still remain the major mechanism of adsorption of LPS.

The inventive method further includes passing the blood through a second group of polymeric particles. The particles of the second group are formed so as to retain cytokines and superantigens. These toxins are electrically neutral proteins. They are smaller than the LPS particles and range in size between 8 and 29 kDa, i.e, in the range of middle molecular weight toxins. The polymer particles of the second group are also hydrophobic in their interior and have a pore size selected so as to provide a close contact of the cytokines and superantigens with the hydrophobic surface of the pores. The polymeric particles of the second group are predominantly mesoporous with the pore size ranging from 2 to 70 nm, preferably from 5 to 50 nm.

The hydrophobic particles of both groups of polymeric particles can be provided with a hydrophilic coating to guarantee biocompatibility of the particles with the human organism, in particular blood. The hydrophilic coating is thin and permeable so as to allow penetration of endotoxins, cytokines and superantigens to the hydrophobic porous core of the particles.

The hydrophobic cores of the particles of the both groups can be composed, for example, of crosslinked polymeric materials prepared by polymerization or copolymerization of the following monomers: styrene, ethylstyrene, α-methylstyrene, divinylbenzene, diisopropenylbenzene, trivinylbenzene, alkyl methacrylate as methyl methacrylate, buthyl methacrylate. The positively charged functional groups covalently bonded to the surface of the pores of the first group of polymeric particles can be selected from the group composed of amino-, methylamino-, ethylamino-, dimethylamino-, diethylamino-, ethanolamino-, diethanolamino-, polyethylenimino-groups, imidazole, histamine, or basic amino acids as lysine, arginine, histidine. The hydrophilic hemocompatible coatings or the shell of the particles of the both groups can be composed for example of the following materials: polyvinylpyrrolidone, polyhydroxyethyl methacrylate, carboxymethylcellulose, polyurethane.

In accordance with the present invention, the first group of polymer particles and the second group of particles can be arranged in a container, for example a cartridge, one after the other. As a result, when blood taken from the patient passes through the first group of polymer particles, endotoxin from blood adheres to the particles of the first group, and thereafter when the blood thusly purified of endotoxin passes through the second group of polymer particles, cytokines and superantigens adhere to the polymer particles of the second group. The blood that passes through the particles of both groups is therefore purified from endotoxin and from cytokines and superantigens, and then returned to the patient. It is of course possible that the blood first passes through the polymer particles from the second group to remove cytokines and superantigens, and thereafter passes through the polymer particles of the first group to remove endotoxin.

Finally it is also possible to provide a mixture of the polymer particles of the first group with the polymer particles of the second group. When the blood is taken from the patient and passes through the mixture of the particles, endotoxin is removed by adherence to the charged hydrophobic surface of the particles of the first group, and the cytokines and superantigens are removed by adherence to the hydrophobic surface of the particles of the second group.

The particles of the first group and the second group can be for example beads, granules, fibers, etc.

The material to be used in the method in accordance with the present invention can be produced as explained in the following examples.

### EXAMPLE 1

In order to produce polymer particles of the first group, into a seven-liter four-necked round-bottom flask equipped with a stirrer, a thermometer and a reflux condenser, is placed the solution of 8.4 g polyvinyl alcohol-type technical grade emulsion stabilizer Aervol 523, 40 g of sodium chloride, and 150 mg of sodium nitrite in four liters of deionized water (aqueous phase). The solution of 260 ml divinylbenzene, 140 ml ethylvinylbenzene, 500 ml n-octane and 2.94 g benzoyl peroxide (organic phase) is then added to the aqueous phase on stirring at room temperature. In 20 min, the temperature is raised to 80° C. The reaction is carried out at 80° C. for 12 hours. After accomplishing the copolymerization, the stabilizer is rigorously washed out with hot water (60 to 80° C.) and the above organic solvents are removed by steam distillation. The beads obtained are filtered, washed with 1000 ml isopropyl alcohol and with deionized water. The polymer is then suspended in three liters of deionized water and supplied at 40° C. with 10 g ammonium persulfate, 10 ml tetramethyl ethylenediamine and finally 8 ml vinylpyrrolidone. The mixture was stirred for 2 hours, the polymer filtered and washed with depyrogenated water. The polymer displayed apparent inner surface area of 300 sq.m/g, total pore volume of 0.85 ml/g, and mean pore diameter of 35 nm.

In order to produce polymer particles of the second group, in a three-liter round-bottom reactor, a mixture of 160 ml divinylbenzene (65% purity), 110 ml toluene, 160 ml iso-octane and 1.12 g benzoyl peroxide (organic phase) was dispersed in a solution of 40 g polyvinylpyrrolidone (MW 40.000), 1.9 g monosodium phosphate, 6.3 g disodium phosphate, 3.9 g trisodium phosphate, and 18 mg sodium nitrite in 1000 ml water. The dispersion was agitated for 19 h at 80° C. After accomplishing the copolymerization, the stabilizer was rigorously washed with hot water and the above organic components were removed by washing the beads with ethanol and pure water. The polymer displayed apparent inner surface area of 650 sq.m/g, total pore volume of 0.95 ml/g, and mean pore diameter of 16 nm.

Then, as explained above, the particles of the both groups are intermixed with one another, or arranged as separate beds one after the other.

### EXAMPLE 2

In order to produce polymer particles of the first group copolymerization was performed as described in Example 1 with the difference that the organic phase contained 20 ml of vinylbenzylchloride, in addition to all the other components, and that the aqueous phase was adjusted to a pH value between 4 and 6. In this way free chloromethyl groups were introduced onto the surface of the porous hydrophobic core of polymer beads. After applying the hemocompatible polyvynylpyrrolidone coating on the surface of the beads, by following the procedure described in Example 1, the material was heated with a 5% solution of diethanolamine. Substitution of surface exposed chloromethyl groups for positively charged diethanolamine groups was achieved in this additional step. The polymer particles of the second group are produced as in Example 1.

When blood with endotoxin and superantigens passes through such a material no measurable amounts of endotoxin and superantigens were found in blood and also the following cytokines were efficiently removed: interlukine 1 L-1-beta, IL-6-alpha, IL-10, and tumor necrose factor TNF alpha.

It will be understood that each of the elements described above, or two or more together, may also find a useful application in other types of methods differing from the types described above.

While the invention has been illustrated and described as embodied in method of treating patients with bacterial infections, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

1. A method of treating serious infections and sepsis caused by infections, comprising the steps of withdrawing blood from a patient; passing the withdrawn blood through a particulate hemocompatible polymer material which includes a first group of macroporous particles which are hydrophobic and positively charged so as to provide adherence of endotoxin to an inner surface of particles of the first group, and also a second group of mesoporous particles which are hydrophobic and are not charged and have a pore size selected so that cytokines and superantigens adhere to an inner surface of the particles of the second group; and thereafter returning the blood back to the patient.

2. A method as defined in claim 1, wherein said particles are particles selected from the group consisting of beads and fibers.

3. A method as defined in claim 1, wherein the macroporous particles of the first group and mesoporous particles of the second group have a hydrophobic porous core part and a hydrophilic coating part providing a biocompatibility.

4. A method as defined in claim 3, wherein the macroporous particles of the first group have a hydrophobic core bearing positively charged groups on the surface of the pores.

5. A method as defined in claim 1, wherein said particles of said first group and said second group are intermixed with one another, said passing including passing through said intermixed bed of particles of said groups.

6. A method as defined in claim 1, wherein said groups of particles are located one after the other, said passing including passing first through the particles of one of said groups, and thereafter through the particles of the other of said groups.
